(19) United States
(12) Patent Application Publication
(10) Pub. No.: US 2017/0363565 A1
(43) Pub. Date: Dec. 21, 2017

(54) Analyte Measurement

(71) Applicant: INSIDE BIOMETRICS LIMITED, Dingwall (GB)

(72) Inventors: Stephen Blythe, Dingwall (GB); Damian Baskeyfield, Dingwall (GB)

(21) Appl. No.: 15/533,780

(22) PCT Filed: Dec. 7, 2015
(86) PCT No.: PCT/GB2015/053736

§ 371 Date: Jun. 7, 2017

(30) Foreign Application Priority Data
Dec. 8, 2014 (GB) 1421816.8

(51) Int. Cl. G01N 27/327; G01N 33/487

(52) U.S. Cl. CPC G01N 27/3274; G01N 27/3272; G01N 33/48707

(57) ABSTRACT

A method for configuring a device to determine a concentration of an analyte uses a plurality of m fluid samples, each having a corresponding known analyte concentration. The method includes, for each sample: generating an output signal from the sample; recording values of the signal over time; and modelling a subset of the values of the signal using n basis functions to obtain n coefficients. Each coefficient is associated with a corresponding basis function, the n basis functions and n coefficients representing the signal for the subset. The method also includes performing a statistical analysis of the m×n coefficients and corresponding known analyte concentrations to determine a set of n parameters from which an analyte concentration can be estimated based on a set of n coefficients obtained for a sample for which the analyte concentration is unknown; and storing the set of n parameters in a memory of a device.

### TECHNICAL FIELD

The present disclosure relates to a method for configuring a device to determine a concentration of an analyte in a fluid sample. The device may be a handheld device such as a meter or other electronic device. In specific embodiments the fluid sample is provided to an electrochemical test device, such as an electrochemical test strip, as used with bodily fluid meters for determining the concentration of an analyte in an individual's bodily fluid sample.

### BACKGROUND

In the field of diagnostic devices as used in the medical device industry, especially those used for analysing blood or other bodily fluid samples, it is often required for users to monitor biometrics such as the levels of certain chemicals, substances, or analytes present for example in their bloodstream. For instance diabetics in particular must regularly monitor the concentrations of glucose in their blood in order to determine if they are in need of insulin or sugar. In order to respond effectively to an individual's need to monitor blood sugar levels, diagnostic devices and kits have been developed over the years to allow an individual to autonomously determine the concentration of glucose in their bloodstream, in order to better anticipate the onset of hyperglycaemia or hypoglycemia and take preventative action as necessary.

Typically the patient will, using a lancing device, perform a finger stick to extract a small drop of blood from a finger or alternative site. An electrochemical test device, which is often a test strip, is then inserted into a diagnostic meter, and the sample is applied to the test strip. Through capillary action, the sample flows across a measurement chamber of the device and into contact with one or more electrodes or similar conductive elements coated with sensing chemistry for interacting with a particular analyte or other specific chemical (for example glucose) in the blood sample. The magnitude of the reaction is dependent on the concentration of the analyte in the blood sample. The diagnostic meter may detect the current generated by the reaction of the reagent with the analyte, and the result can be displayed to the user.

Typically, such electrochemical test devices have a counter/reference electrode and one or more working electrodes. Sensing chemistry is used which is typically tailored to the particular analyte of interest. For example, when measuring the concentration of glucose in a sample, a glucose oxidase or a glucose dehydrogenase enzyme can be used in conjunction with a mediator such as potassium ferricyanide. The skilled person will understand that different electrochemical test devices, electrode arrangements and sensing chemistry may be used.

It is important that the reading output by a meter can be relied upon so that, if necessary, appropriate action may be taken. If the reading is erroneous and the user acts upon the erroneous reading, any action taken (e.g. the administration of insulin or sugar) could be detrimental to the user's health. Erroneous readings can arise not only if the strip is damaged (which could affect the flow rate of the fluid sample across the measurement chamber) or if the meter itself is damaged, but also if other components of the fluid sample affect the output reading of the meter.

One notable component of a fluid sample which may affect the reading output by a meter is the red blood cells. This is measured by the haematocrit level, also known as packed cell volume (PCV) or erythrocyte volume fraction (EVF), which measures the volume percentage of red blood cells in a sample. Typically the haematocrit is around 45% for men and around 40% for women. If, for example the haematocrit is higher than expected (i.e. there are more red blood cells in the fluid sample than expected) then it is likely that the concentration of the analyte under study is lower in the volume than expected. If the haematocrit is lower than expected (i.e. the red blood cell count in the sample is lower than expected) then it is likely that the concentration of the analyte under study in the sample may be higher than expected.

There therefore remains a need in the art to configure a device in such a way as to be less sensitive to non-analyte components in a fluid sample.

### SUMMARY

In accordance with a first aspect, there is provided a method for configuring a device to determine a concentration of an analyte. The method uses a plurality of m fluid samples, each fluid sample of the m fluid samples having a corresponding known analyte concentration. The method comprises, for each fluid sample of the m fluid samples, generating an output signal from the fluid sample. The method further comprises, for each fluid sample of the m fluid samples, recording values of the output signal over time. The method further comprises, for each fluid sample of the m fluid samples, modelling at least a subset of the recorded values of the output signal using n basis functions to obtain n coefficients, each coefficient being associated with a corresponding basis function. The n basis functions and n coefficients represent the output signal for the subset. The method further comprises performing a statistical analysis of the m×n coefficients and corresponding known analyte concentrations to determine a set of n parameters from which an analyte concentration can be estimated based on a set of n coefficients obtained for a fluid sample for which the analyte concentration is unknown. The method further comprises storing the set of n parameters in a memory of one or more devices.

Advantageously, by modelling at least a subset of the recorded values of the output signal using n basis functions to obtain n coefficients, each output signal can be easily represented by the series of coefficients and can be compared with the coefficients established for other fluids samples. Further advantageously, by performing a statistical analysis of the m×n coefficients and corresponding known analyte concentrations, errors introduced by non-analyte components of a fluid sample can be accounted for. Accordingly, once a determined set of parameters has been stored in a memory of one or more devices, an estimate of the analyte concentration in a fluid sample for which the analyte concentration is unknown can be obtained, the estimate being less sensitive to non-analyte components in the sample such as extra red blood cells (RBCs).

Generating an output signal from each fluid sample may comprise applying an input to the fluid sample to generate the output signal. The input may be an input signal. Applying an input to the fluid sample may comprise applying a potential difference across the fluid sample. The output signal may be a transient current.

The basis functions may be orthogonal basis functions. Advantageously, by using orthogonal basis functions, less computational time is required for modelling recorded values of an output signal using the n basis functions to obtain the n coefficients. In some embodiments the basis functions are orthogonal on the range [0, 1]. The basis functions may be shifted Legendre polynomials. Advantageously, the shifted Legendre polynomials are orthogonal with respect to a weighting function of unity on the support. Accordingly, this leads to a reduced overhead in computing the corresponding coefficients.

The value of n may be greater than or equal to 3 and less than or equal to 10. The value of n may be greater than or equal to 1 and less than or equal to 20. The value of n may be greater than 20. The higher the number of basis functions used, the greater the accuracy with which the output signal can be modelled, although this leads to an increase in the computation time of the n coefficients. The modelling at least a subset of the recorded values of the output signal over the time period using n basis functions may comprise calculating a least-squares best fit of the recorded values to the n basis functions. Accordingly, the output signal of a fluid sample may be modelled by the least-squares best fit of the recorded values of the output signal to the basis functions.

Optionally, the performing of a statistical analysis of the m×n coefficients and corresponding known analyte concentrations comprises performing a regression analysis of the m×n coefficients and corresponding known analyte concentrations.

Recording values of the output signal may comprise taking time based measurements of the output signal. In some embodiments a large number of values are recorded. For example, a number of values that is greater than or equal to 100 and is less than or equal to 1000 may be recorded. The time-based measurements may optionally be recorded at a frequency that is greater than or equal to 10 Hz and less than or equal to 1000 Hz.

Optionally, modelling at least a subset of the recorded values of the output signal comprises modelling all recorded values of the output signal. Optionally, modelling at least a subset of the recorded values of the output signal comprises modelling a portion of the recorded values. Modelling at least a subset of the recorded values of the output signal may further comprise modelling a second portion of the recorded values. The portion of the recorded values and the second portion of the recorded values may overlap. Alternatively, the portion of the recorded values and the second portion of the recorded values may not overlap.

Each fluid sample of the plurality of m fluid samples may comprise a non-analyte component, the presence of which affects the output signal generated for the fluid sample. There may be a variation in the concentration of the non-analyte component across the plurality of m samples. The statistical analysis of the m×n coefficients and corresponding known analyte concentrations may correct for the variation in the concentration of the non-analyte component across the plurality of m samples. For configuring a device, the concentration of the non-analyte component may substantially be known for each sample of the plurality of m samples. The non-analyte component may comprise red blood cells.

Each fluid sample may be a biological fluid sample. The biological sample may be, for example, a blood sample, an interstitial fluid sample, or a plasma sample.

A large number of fluid samples may be used. That is, m may be greater than or equal to 500 and less than or equal to 1000. When the number of fluid samples is large, a better statistical analysis can be performed to arrive at the parameters from which an analyte concentration can be estimated based on a set of n coefficients obtained for a fluid sample for which the analyte concentration is unknown.

The analyte may be glucose. The analyte may be one of lactate, glycerol, cholesterol, or a ketone such as β-hydroxybutyrate.

In accordance with a second aspect, there is provided an apparatus for configuring a device to determine a concentration of an analyte. The apparatus comprises circuitry for generating an output signal from a fluid sample. The apparatus further comprises a memory storing instructions to perform any method described above. The apparatus further comprises a processor configured to perform the instructions stored in the memory.

The output signal may be a transient current. The apparatus may further comprise circuitry for applying an input to the fluid sample to generate the output signal. The circuitry for applying an input signal to the fluid sample may comprise circuitry for applying a potential difference across the fluid sample. The apparatus may be configured to receive an electrochemical test device for receiving the fluid sample.

In accordance with a third aspect, there is provided a machine readable medium having instructions stored thereon, the instructions being configured such that when read by a machine the instructions cause any of the methods above to be carried out.

In accordance with a fourth aspect, there is provided a method of determining a concentration of an analyte in a fluid sample for which the analyte concentration is unknown. The method comprises generating an output signal from the fluid sample. The method further comprises recording values of the output signal over time. The method further comprises modelling at least a subset of the recorded values of the output signal using n basis functions to obtain n coefficients for the fluid sample. Each of the coefficients is associated with a corresponding basis function. The n basis functions and the n coefficients represent the output signal for the subset. The method further comprises using a predetermined set of n parameters to estimate the analyte concentration from the n coefficients.

Generating an output signal from each fluid sample may comprise applying an input to the fluid sample to generate the output signal. The input may be an input signal. Applying an input to the fluid sample may comprise applying a potential difference across the fluid sample. The output signal may be a transient current.

Using a predetermined set of n parameters to estimate the analyte concentration from the n coefficients may comprise, for each of the n parameters, multiplying the parameter by a corresponding one of the n coefficients to form a combined product. The combined products may then be added to provide an estimate of the concentration of the analyte in the sample.

In accordance with a fifth aspect, there is provided a device for determining a concentration of an analyte in a fluid sample for which the analyte concentration is unknown. The device comprises circuitry for receiving an output signal generated from a fluid sample. The device further comprises a memory storing instructions to perform a method of determining a concentration of an analyte in a fluid sample for which the analyte concentration is unknown, such as that described above. The device further comprises a processor configured to perform the instructions stored in the memory. The output signal may be a transient current.

The device may be configured to receive the output signal from a separate component which generates the signal from the fluid sample. The separate component may be or comprise an electrochemical test device. The separate component may comprise a patch, for example. Electrochemical test devices such as patches typically comprise a subcutaneous fluid extraction set and sensing chemistry for interaction with the analyte. The separate component may be a monitoring component which transmits the output signal to the device, either wirelessly or through a wired connection. The separate component may comprise a continuous monitoring device or a semi-continuous monitoring device.

The device may be configured to directly connect to, or receive an electrochemical test device for receiving the fluid sample. The electrochemical test device may comprise a test strip, for example. Electrochemical test devices such as test strips comprise a measurement chamber and one or more electrodes with sensing chemistry for interacting with the analyte. The electrochemical test device may be configured for one-time use. That is, the electrochemical test device may be disposable.

Whether directly connected to a device, or operating as a separate component, the electrochemical test device may be configured for testing the concentration of multiple analytes. The device may be configured to carry out the above method for multiple analyte components of the sample.

The device, or the separate component, whichever may be the case, may further comprise circuitry for applying an input signal to the fluid sample to generate the output signal. The circuitry for applying an input to the fluid sample may comprise circuitry for applying a potential difference across the fluid sample.

The device may be a meter. The device may be any type of electronic device, such as a smart phone, computer, personal digital assistant or other electronic device. The device may comprise one or more distributed devices, for example, one or more distributed computer systems on a network.

In accordance with a sixth aspect, a machine readable medium having instructions stored thereon is provided. The instructions are configured such that when read by a machine the instructions cause a method of determining a concentration of an analyte in a fluid sample for which the analyte concentration is unknown, such as the method described above, to be carried out.

### BRIEF DESCRIPTION OF THE FIGURES

FIG. 1 shows a strip-meter system;

FIG. 2 shows an example of a transient current;

FIG. 3 shows an apparatus for configuring a meter to determine a concentration of an analyte;

FIG. 4 shows an example computer apparatus that may be used for configuring a meter to determine a concentration of an analyte;

FIG. 5 shows a flowchart of a first part of a method for configuring a device to determine a concentration of an analyte;

FIG. 6 shows a flowchart of a second part of a method for configuring a device to determine a concentration of an analyte;

FIG. 7 shows a flowchart of a method of determining a concentration of an analyte in a fluid sample for which the analyte concentration is unknown;

FIG. 8 is a graph showing the extent to which glucose values can be reconstructed for each of a series of samples by carrying out the described method;

FIG. 9 shows a graph of current against time for transient currents generated by applying a potential difference across 660 fluid samples;

FIG. 10 shows the result of calibrating the test data from FIG. 9 with the end current at nominal (42%) haematocrit, and applying this calibration to get an estimated glucose reading; and

FIG. 11 shows the predicted (estimated) glucose values after carrying out the described method.

### DETAILED DESCRIPTION

The disclosed embodiments provide an improved method for configuring a device to determine a concentration of an analyte in a fluid sample. Whilst various embodiments are described below, the claims are not limited to these embodiments, and variations of these embodiments may well fall within the scope of the claims.

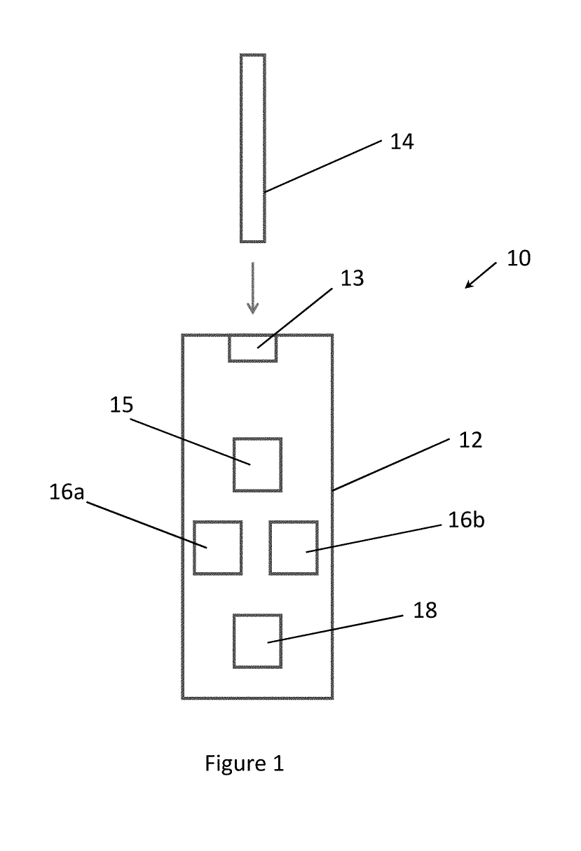

FIG. 1 shows a strip-meter system 10 according to an embodiment. System 10 comprises a meter 12 for receiving an output signal from an electrochemical test device such as electrochemical test strip 14. Electrochemical test strip 14 comprises one or more electrodes (not shown) and a counter/reference electrode, each of the working electrodes having a reagent coated thereon for reacting with a fluid sample to be applied to electrochemical test strip 14. The counter/reference electrode may also have a reagent coated thereon. Meter 12 comprises receiving means 13 for receiving electrochemical test strip 14 and applying a potential difference to the working electrode(s) and the counter/reference electrode.

Meter 12 further comprises processing circuitry 15 for carrying various functions relating to the operation of meter 12. For example, processing circuitry 15: controls operation of receiving means 13 so as to control application of a potential difference between the working electrodes and the counter/reference electrode; processes one or more output signals generated at test strip 14; controls the display of messages on display 18; etc. Meter 12 further comprises the first second memory storages 16a and 16b. Although two memory storages are shown, in other embodiments the memory storages may be combined to form a single memory storage, or meter 12 may comprise more than two memory storages. Meter 12 also comprises a display 18 for displaying readouts of measurements taken by meter 12.

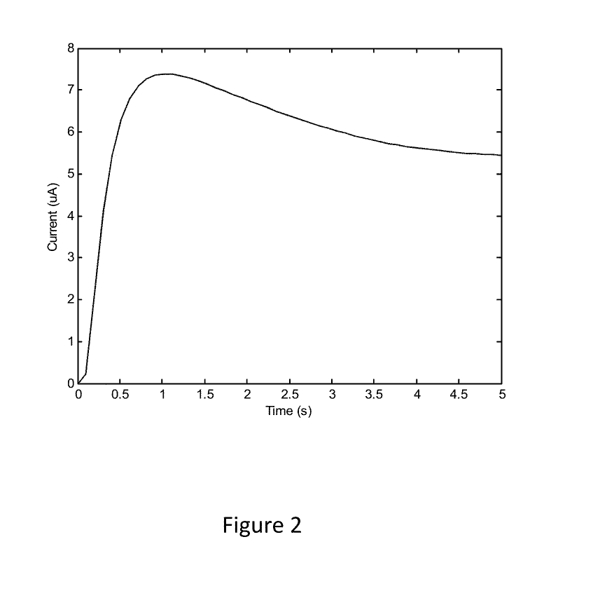

An electrochemical test device may provide a fluid sample having an unknown analyte concentration to meter 12. Applying a potential difference across the fluid sample may generate an output signal having a profile much like that shown in FIG. 2. In FIG. 2, the output signal is a transient current (shown in units of micro-Amperes) generated by applying the potential difference across a blood sample. Values of the transient current are shown over a 5 second period.

By analysing the output signal generated from applying the potential difference across a fluid sample, one may obtain an estimate of the concentration of an analyte in the fluid sample. In existing meters, non-analyte components of the fluid sample may affect the output signal generated and thereby lead to an inaccurate estimate of the concentration of the analyte in the fluid sample. Accordingly methods and apparatus for configuring a device to determine a concentration of an analyte will now be described.

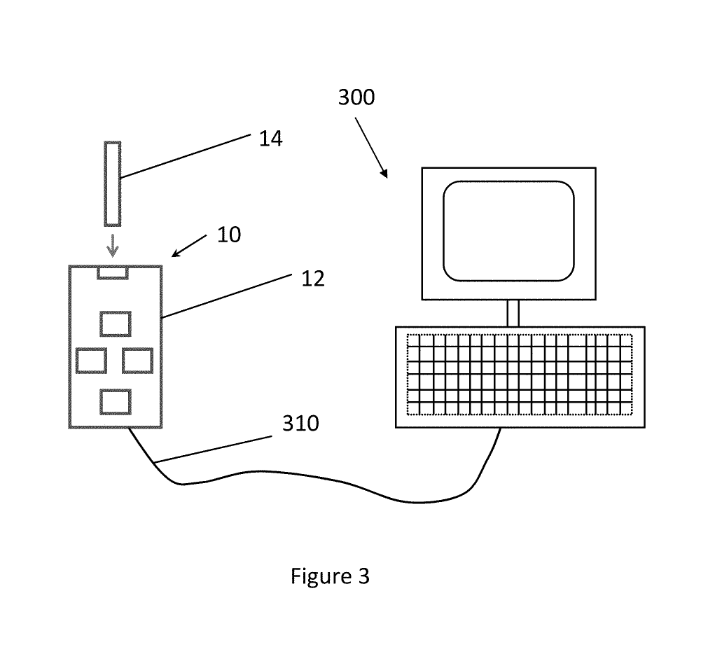

An apparatus for configuring a meter to determine a concentration of an analyte will now be described in connection with an embodiment. FIG. 3 illustrates such an embodiment. In this embodiment, a strip-meter system 10, such as that shown in FIG. 1, is connected via a connection 310 to a computer system 300. The strip-meter system comprises a meter 12 for reading a plurality of electrochemical test devices such as electrochemical test strip 14. The plurality of electrochemical test devices may be read sequentially, simultaneously or in any other suitable manner. Each of the plurality of electrochemical test devices provides a fluid sample to the meter, each fluid sample having a known concentration of an analyte. By applying a potential difference across a fluid sample, an output signal such as a transient current is generated. At the computer system 300, the output signal is modelled using a number of basis functions. An analysis is performed on the modelled data and the known analyte concentrations of the fluid samples to provide a set of parameters which can be used to configure a meter to determine a concentration of the analyte for a fluid sample for which the analyte concentration is unknown.

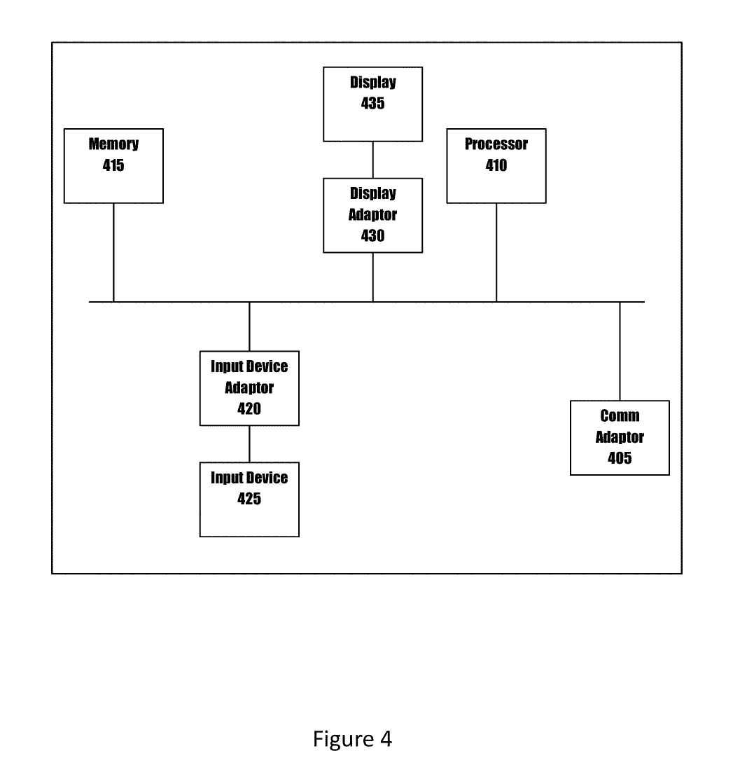

FIG. 4 illustrates an example computer apparatus that may be used as a part of the apparatus for configuring a meter to determine a concentration of an analyte, although other architectures may be used as will be appreciated by the skilled person. Referring to FIG. 4, the computer apparatus comprises a communications adaptor 405, a processor 410 and a memory 415. The computer apparatus also comprises an input device adaptor 420 for communicating with an input device 425. The computer further comprises a display adaptor 430 for operation with a display 435.

The processor 410 is configured to receive data, access the memory 415, and to act upon instructions received either from said memory 415 or said communications adaptor 405. The communication adaptor 405 is configured to receive data and to send out data.

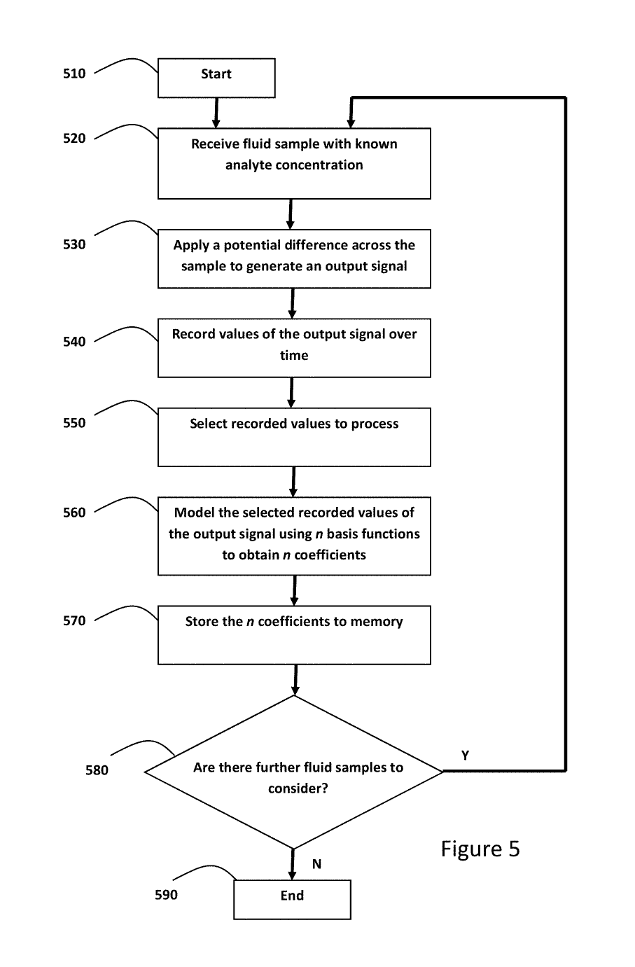

A first part of a method for configuring a device to determine a concentration of an analyte will now be described in connection with an embodiment. In this embodiment, the fluid sample is a blood sample provided to the apparatus via an electrochemical test device such as an electrochemical test strip. The analyte under consideration is glucose. It should be noted that FIG. 5 shows an example method, and the order of the steps may be changed without departing from the scope. The method may also comprise a lower or greater number of steps.

At step 510 the method begins. At step 520 the apparatus receives an electrochemical test device and a blood sample is obtained, the blood sample having a known glucose concentration. The blood sample is applied to the electrochemical test device.

At step 530 processing circuitry controls the application of a potential difference between a working electrode and a counter/reference electrode of the apparatus, and thereby controls the application of a potential difference across the blood sample, which generates an output signal, in this case a transient current. At step 540 the transient current is recorded over time. In particular, at 1000 points in time, values of the transient current are recorded and stored to memory. For example, if the transient current is recorded over a 5 second period, then the time interval between measurements is 5/1000 seconds.

At step 550, recorded values are selected for processing. The selected recorded values may comprise all of the recorded values for the sample at step 540. Alternatively only a portion of the recorded values may be selected.

For example, if at step 540 the transient current is recorded for 5 s, then at step 550, a selection may be made to only analyse the recorded values that occurred between the 3 s and 5 s times. Accordingly, in this case, the time period over which the selected values were recorded is only a portion of the time over which the values of the transient current were recorded, and a portion of all the recorded transient current values is analysed.

At step 560 the selected recorded values of the transient current are modelled using n basis functions to obtain n coefficients, each coefficient being associated with a corresponding basis function, the n basis functions and n coefficients representing the transient current over the time period.

The current measured at each time tin a transient may be denoted as I(t). This signal contains contributions from the analyte of interest, other sources of systematic and unwanted signal such as haematocrit, and measurement noise.

It is convenient to represent the signal as the sum of known basis functions, separating this from the representation of the noise. A suitable set of basis functions are the shifted Legendre polynomials, where the j^{th} shifted Legendre polynomial can be found by:

$${\tilde{P}}_{j} ( x ) = {( - 1 )}^{j} {\sum }_{l = 0}^{j} ( \begin{matrix}j \\ l\end{matrix} ) ( \begin{matrix}j + l \\ l\end{matrix} ) {( - x )}^{l} \qquad ( EQUATION 1 )$$

where x is greater than or equal to 0 and less than or equal to 1. The index j is an integer greater than or equal to zero. Here, (ₗ^{j}) represents a binomial coefficient.

Additionally the shifted Legendre polynomials are orthogonal on the range [0, 1]. That is,

$${\int }_{0}^{1} {\tilde{P}}_{j} ( x ) {\tilde{P}}_{k} ( x ) dx = \frac{1}{2 j + 1} {δ}_{jk} \qquad ( EQUATION 2 )$$

where δⱼₖ denotes the Kronecker delta.

The time period is modelled such that the time t is scaled to be between 0 and 1, i.e. x=t/tₘₐₓ, where tₘₐₓ is the highest value of time t over the time period.

Using the shifted Legendre polynomials, and normalising the times at which the selected recorded values were made so as to be scaled between 0 and 1, the transient current can be represented as:

$$I ( x ) = {\sum }_{j = 0}^{∞} {β}_{j} {\tilde{P}}_{j} ( x ) + ɛ \qquad ( EQUATION 3 )$$

where \tilde{P}ⱼ(x) is the j^{th} shifted Legendre polynomial, ε is noise with zero mean at each scaled time x and βⱼ is a coefficient. In Equation 3, a high level of accuracy can be achieved by summing index j from 0 to some finite value n.

Referring back to step 560 of FIG. 5, the selected recorded values of the transient current can be modelled using the first n shifted Legendre polynomials, i.e. the shifted Legendre polynomials \tilde{P}₀(x), \tilde{P}₁(x) . . . \tilde{P}ₙ₋₁(x). It is noted that \tilde{P}₀(x)=1 for any value of x.

A least-squares fit of the selected recorded values to the shifted Legendre polynomials minimizes the integral S, where

$$S = {\int }_{0}^{1} {( I ( x ) - {\sum }_{j = 0} {β}_{j} {\tilde{P}}_{j} ( x ) )}^{2} dx . \qquad ( EQUATION 4 )$$

Due to the orthogonal nature of the shifted Legendre polynomials, the best-fit parameter values can be obtained independently of each other according to

βⱼ=(2j+1)∫₀¹\tilde{P}ⱼ(x)I(x)dx. (EQUATION 5)

Accordingly the order of the fit can be increased until sufficient accuracy has been achieved, without changing the lower order coefficient estimates. This is in contrast to fitting with standard polynomial models where all of the coefficients must be re-estimated if the order of polynomial is changed. When the fluid sample is blood and the analyte for which a concentration is to be measured is glucose, the inventors have found that for n in the region of 7 or 8, good results are acquired.

The n coefficients may be found from the recorded values of the transient current by:

$$\begin{matrix}( \begin{matrix}{β}_{0} \\ ⋮ \\ {β}_{n - 1}\end{matrix} ) = {( {X}^{T} X )}^{- 1} {X}^{T} ( \begin{matrix}I ( {x}_{0} ) \\ ⋮ \\ I ( {x}_{n - 1} )\end{matrix} ) & ( EQUATION 6 ) \\ where & \\ X = ( \begin{matrix}{\tilde{P}}_{0} ( {x}_{0} ) & … & {\tilde{P}}_{n - 1} ( {x}_{0} ) \\ ⋮ & ⋱ & ⋮ \\ {\tilde{P}}_{0} ( {x}_{n - 1} ) & … & {\tilde{P}}_{n - 1} ( {x}_{n - 1} )\end{matrix} ) . & ( EQUATION 7 )\end{matrix}$$

In equations 6 and 7, each of the values xⱼ represents a (normalised) time at which a measurement of the current was made.

By performing the above method, a set of n coefficients (the values βⱼ) are found for the transient current generated for the fluid sample. The n coefficients and the n basis functions together represent the transient current generated by applying the voltage across the sample.

At step 570 the coefficients are stored to a memory. After storing the coefficients to memory on the apparatus, if there are further samples to process (step 580) then the method loops back to step 520 at which point another fluid sample is received by the device. There are m fluid samples to process. Once all m blood samples have been processed (step 580) then the method concludes at step 590. When method step 590 is reached, then for all m samples tested a set of n β coefficients will have been stored in the memory of the apparatus. Additionally the known glucose concentrations for each sample are stored in the memory of the apparatus for later reference.

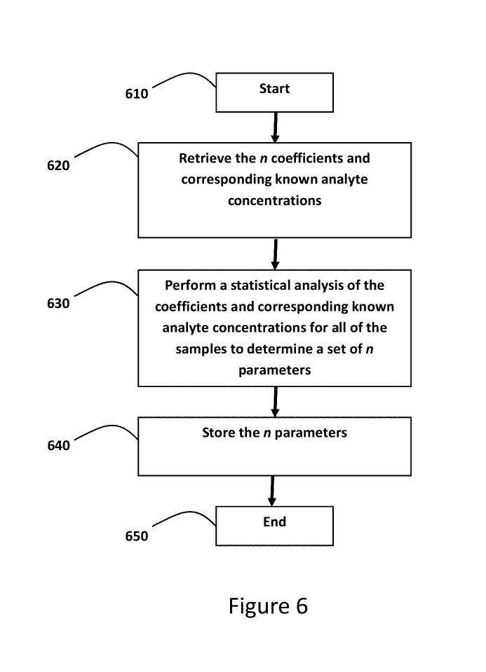

After the β coefficients have been calculated for each of the blood samples, a method such as that illustrated in the flowchart of FIG. 6 can be carried out so as to configure a meter to determine a concentration of glucose in further blood samples. At step 610 the method begins.

At step 620 the n coefficients for each blood sample and the corresponding known analyte concentration values are retrieved from the memory of the apparatus.

At step 630 a statistical analysis of all of the m×n calculated coefficients and corresponding known analyte concentrations is performed in order to determine a set of parameters from which an analyte concentration can be estimated based on a set of coefficients obtained for a blood sample for which the glucose concentration is unknown. In this embodiment, the statistical analysis is performed by carrying out a least squares regression of the data. By performing a regression analysis on the data, a set of n parameters, cⱼ are calculated (j=0 . . . n−1). The set of parameters may be used to obtain an estimate of the concentration of glucose in further blood samples for which glucose concentration is unknown.

The parameters cⱼ may be calculated from

$$\begin{matrix}( \begin{matrix}{c}_{0} \\ ⋮ \\ {c}_{n - 1}\end{matrix} ) = {( {Y}^{T} Y )}^{- 1} {Y}^{T} ( \begin{matrix}{g}^{( 1 )} \\ ⋮ \\ {g}^{( m )}\end{matrix} ) & ( EQUATION 8 ) \\ where & \\ Y = ( \begin{matrix}{β}_{0}^{( 1 )} & … & {β}_{n - 1}^{( 1 )} \\ ⋮ & ⋱ & ⋮ \\ {β}_{0}^{( m )} & … & {β}_{n - 1}^{( m )}\end{matrix} ) . & ( EQUATION 9 )\end{matrix}$$

In equations 8 and 9, the superscript (j) indicates the j^{th} sample. For example, β₀⁽¹⁾ is the zeroth coefficient calculated for the first of the m fluid samples. The value g^{(j)} is the known glucose concentration value of the j^{th} sample.

At step 640 the parameters, cⱼ are stored in a memory. The parameters are input into a memory of one or more devices for future use. At step 650 the method ends.

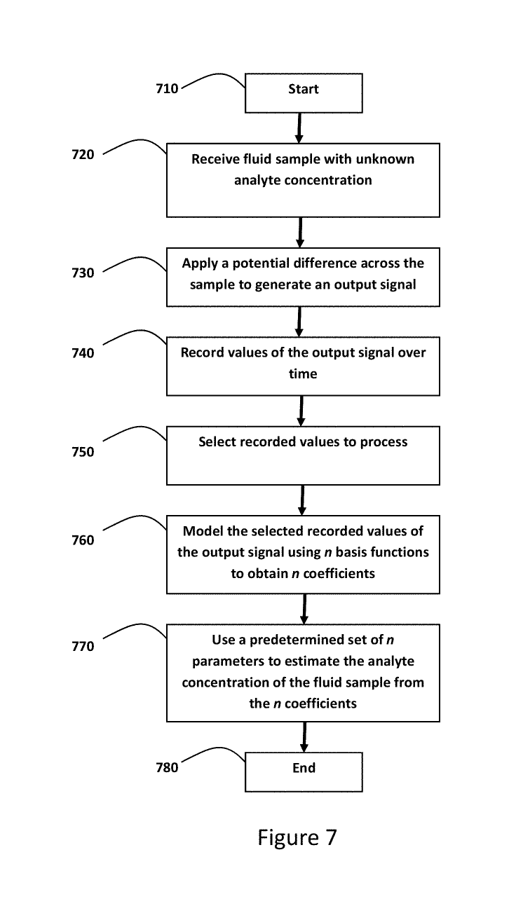

FIG. 7 is a flow chart showing a method of determining a concentration of glucose in a blood sample for which the glucose concentration is unknown, the method using the parameters, cⱼ, stored in a memory of a device. The device may be, for example, a meter such as meter 12 of FIG. 1. At step 710 the process begins.

At step 720 an electrochemical test device with a blood sample having an unknown glucose concentration is received by the meter. The electrochemical device is used to provide a blood sample to the meter.

At step 730 a potential difference is applied across the blood sample in order to generate an output signal such as a transient current. Values of the transient current are recorded over time in a memory of the meter (step 740).

At step 750, recorded values are selected for processing, the recorded values corresponding to a particular time period.

At step 760, at least a subset of the recorded values of the transient current are modelled using the n basis functions to obtain n coefficients for the blood sample, each coefficient being associated with a corresponding basis function, the n basis functions and n coefficients representing the transient current for the subset. The n basis functions that are used are the same n basis functions used in step 560 of FIG. 5 and discussed above. That is, the first n shifted Legendre polynomials are used as basis functions. The n coefficients \tilde{β}ⱼ (j=0 . . . n−1) are found by a least-squares best fit of the recorded values of the blood sample to the first n shifted Legendre polynomials in the same way as discussed above.

Once the n coefficients \tilde{β}ⱼ have been calculated, at step 770, the predetermined set of parameters, cⱼ, stored in the memory of the meter are retrieved and are used in conjunction with the calculated n coefficients to estimate the glucose concentration of the blood sample. That is, the glucose concentration estimate gₑₛₜ is found by:

$${g}_{est} = {\sum }_{j = 0}^{n - 1} {c}_{j} {\tilde{β}}_{j} . \qquad ( EQUATION 10 )$$

At step 780 the process ends.

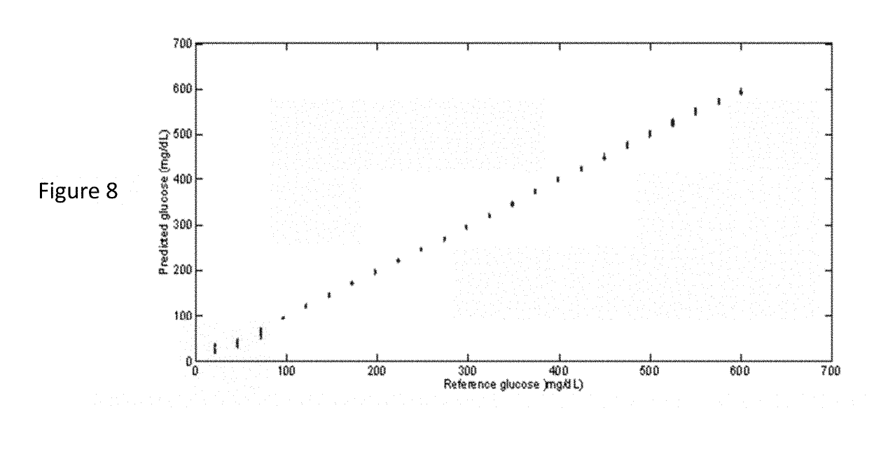

FIG. 8 is a graph illustrating the improved accuracy obtained by an embodiment. Transient currents were generated from a strip simulator for plasma glucose in the range 20 to 600 mg/dL, and haematocrit in the range 20% to 60%. For each generated transient current (576 in total) the shifted Legendre polynomials were fitted up to order 6. That is, for each of the transient currents 50 values were recorded. These recorded values were then fit to the shifted Legendre polynomials up to sixth order using a least-squares analysis. The matrix Y used in the subsequent least-squares regression analysis has 576 rows (observations) and 7 columns (the β coefficients): there are seven columns because the 0^{th} polynomial, \tilde{P}₀(x), is also considered, and is always equal to 1. Applying regression with matrix Y of predictors, fitting to the glucose levels input into the simulator, across all haematocrit values gave the vector of parameters C shown below.

| Parameter | Value |
|---|---|
| c₀ | 0.1476 |
| c₁ | −0.1444 |
| c₂ | 0.1412 |
| c₃ | −0.1261 |
| c₄ | 0.1266 |
| c₅ | −0.0752 |
| c₆ | 0.2527 |

FIG. 8 shows the extent to which the glucose values can be reconstructed for each of the samples. Along the x axis the known glucose values for each of the tested samples are shown. Along the y axis the estimated glucose values are shown for each of the tested samples, the estimated values calculated using the methods disclosed above. That is, after calculating the parameters cⱼ shown in the table, the estimated glucose value for each sample was then calculated using Equation 10.

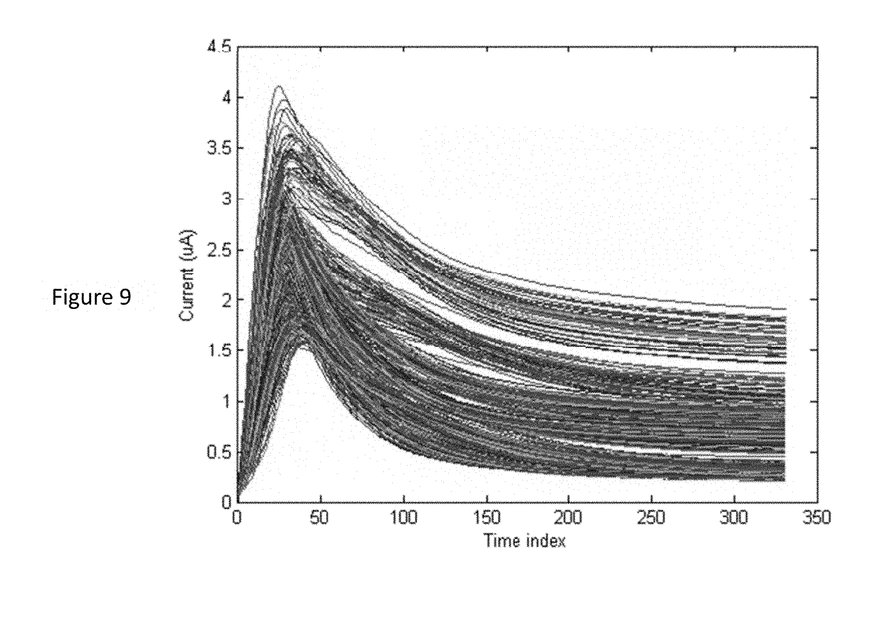

Data from test strips tested with glucose was explored to extend the technique from the model to real test strips. A batch of glucose test strips was produced and tested with a combination of samples comprising five haematocrit levels (20, 30, 42, 50 and 60%) and five glucose levels (50, 100, 200, 300 and 500 mg/dL) test. Accordingly there were 25 sets of glucose/haematocrit combinations. FIG. 9 shows a graph of current against time for the transient currents generated by applying a potential difference across the samples. In particular, FIG. 9 shows m=660 transient currents after error trapping. The recording time for the transient current was 5 seconds and 330 measurements of the current were made. Accordingly the time index along the x axis counts the number of measurements made. The time interval between each measurement is 5/330 seconds. As can be seen from the graph there is a large variation in transient current measured. The large variation in transient currents is due, at least in part, to the variation in the haematocrit and any other non-analyte components etc. across all the samples.

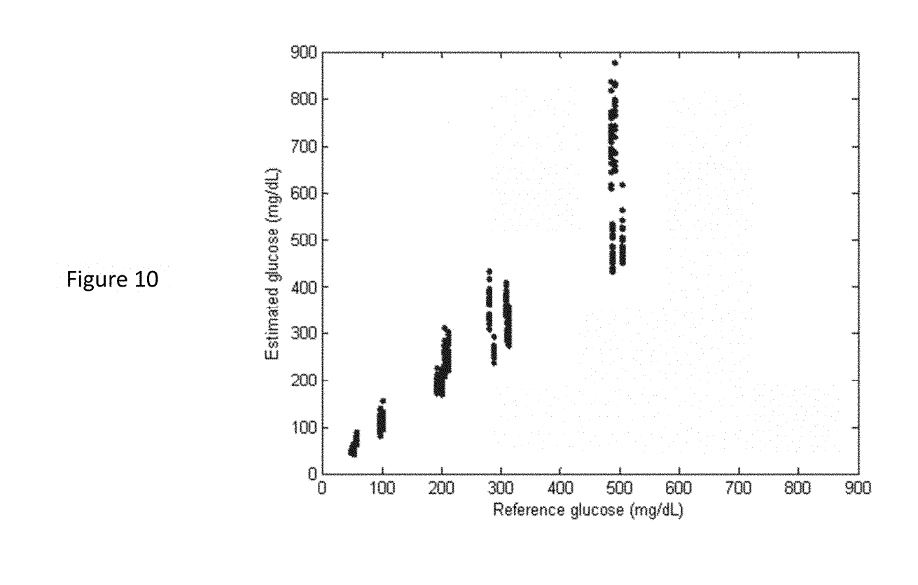

FIG. 10 shows the result of calibrating the test data from FIG. 9 with the end current (here 5 seconds) at nominal (42%) haematocrit, and applying this calibration across all five glucose levels and all five haematocrit levels to get an estimated glucose reading. The large degree of inaccuracy that appears progressively at higher glucose is the result of haematocrit (non-analyte component) sensitivity.

Applying orthogonal polynomials to this data, it is also clear that greatest variation between strips is at earlier times. Hence the polynomials are applied not over the entire 0 s to 5 s range, but over a more stable subset, for example 1.5 to 5 s by way of illustration; other ranges may be chosen.

Following the procedure above, using in this example shifted Legendre polynomials up to order 7, gives the predictor coefficients

| Parameter | Value |
|---|---|
| c₀ | 0.0391 |
| c₁ | 0.2084 |
| c₂ | 0.7222 |
| c₃ | 1.2407 |
| c₄ | 0.6305 |
| c₅ | 0.1562 |
| c₆ | 7.2670 |
| c₇ | 0.1357 |

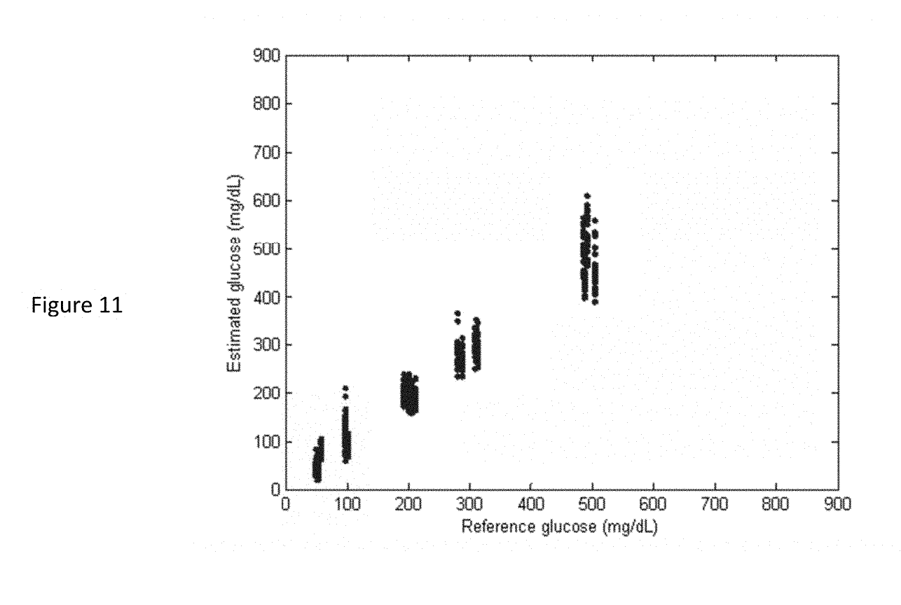

FIG. 11 shows the predicted (estimated) glucose values. As can be seen from the figure when compared with FIG. 10, the large haematocrit inaccuracy has been reduced.

Variations of the described embodiments are envisaged, and the features of the disclosed embodiments can be combined in any way.

The fluid sample may be a biological fluid. For example, the biological fluid may be blood, or may be interstitial fluid, or may be plasma. The analyte may be any analyte found in the fluid sample. For example, the analyte may be glucose, lactate, glycerol, cholesterol, or a ketone such as β-hydroxybutyrate.

The non-analyte component may comprise red blood cells or, when the fluid is blood, any other component of blood which will affect the measurement of the output signal and, in turn, the determined concentration of an analyte in a sample. For example, the non-analyte component may comprise cells, platelets or other cellular components.

The methods and apparatus described above may be used with any suitable electrochemical test device, such as a test strip or a patch. The electrochemical test device may, for example, be suitable for testing for multiple analytes.

When a multi-analyte test device is available, the disclosed methods for configuring a device to determine a concentration of an analyte may be used to configure the device to determine concentrations of multiple analytes. The disclosed methods of determining a concentration of an analyte in a fluid sample for which the analyte concentration is unknown may be extended to determine concentrations of multiple analytes in the fluid sample.

Output signals may be transient currents. The generating of an output signal may comprise applying an input to the fluid sample, such as applying a potential difference across the sample. To one skilled in the art, it would be apparent that the output signal may comprise any suitable signal such as a voltage or other electrical characteristic. For example, in the described embodiments, a potential difference is applied to a fluid sample and values of a transient current are recorded. However, a current input may be applied as an input signal and a voltage output signal may be recorded. Other output signals may be associated with, for example, capacitance or impedance.

In the described examples, the basis functions used were shifted Legendre polynomials. However, the basis functions may be any suitable basis functions. The basis functions may be part of an orthogonal set. Although shifted Legendre polynomials have been discussed above, other orthogonal polynomials may be used, such as any of the classical orthogonal polynomials including Hermite polynomials, Laguerre polynomials, Jacobi polynomials (including as a special subset the Gegenbauer polynomials), Chebyshev polynomials, and Legendre polynomials. Any number of basis functions may be used for determining coefficients for the fluid samples. Good accuracy has been found by using seven or eight shifted Legendre polynomials, but for better modelling of data higher orders of polynomials may be used. Typically n is greater than or equal to 3 or less than or equal to 10. However, n may be any suitable value. For example, n could be 1 or 2, particularly when the modelling of recorded values of an output signal is over a small portion of the all recorded values for a sample. In some cases, for example when the entire output signal for a fluid sample is modelled, a high number of basis functions may be required. For example, n may be 20 or higher.

In the described examples, in order to model at least a subset of the recorded values of the output signal using n basis functions to obtain n coefficients, a least-squares best fit of the recorded values to the basis functions was carried out. However any other suitable method for modelling recorded values of the transient current using basis functions may be used. For example, all of the recorded values for a sample may be sub-divided into k>0 intervals, which can be overlapping. Within each subinterval time may again be scaled to give a scaled time x in the range [0, 1], and one or more polynomials can be fitted to provide β coefficients for the interval. The polynomials in any method need not be of a specific range of orders.

Situations are envisaged in which the time period over which a subset of the recorded values are modelled using n basis functions, is only a portion of the total time used for recording values of the transient current. In this scenario by considering only a small subset of the total number of recorded values, a set of parameters from which an analyte concentration can be estimated based on a set of n coefficients obtained for a fluid for which the analyte concentration is unknown may be determined that represent the particular time period. The behaviour of the transient current outside of that time period may be inferred from the subset of values recorded during the time period.

Additionally, modelling at least a subset of the recorded values of the output signal may comprise modelling a portion (or first portion) of the recorded values. The modelling at least a subset of the recorded values may further comprise modelling a second portion of the recorded values. The first and second portions of recorded values may or may not overlap. The first portion of recorded values may be modelled by substantially fitting the values to a first set of basis functions. The second portion of recorded values may be modelled by substantially fitting the values to a second set of basis functions, and the second set of basis functions may or may not be the same set as the first set of basis functions. As an example, a first portion of recorded values may be modelled using a basis functions from a first set of basis functions and a second portion of recorded values may be modelled using b basis functions from a second set of basis functions, where n=a+b. Of course, further portions of the recorded values may be modelled.

Different inputs may be applied for better characterisation of a fluid sample. For example, a set of fixed potential differences may be applied to a fluid sample. A smoothly changing potential difference may be applied to a sample. Any suitable interrogating waveform may be used. Accordingly, modelling a portion of the recorded values may comprise modelling recorded values that correspond to a particular input being applied to a fluid sample.

In the described examples, a regression analysis has been performed on the m×n coefficients. However, any suitable statistical analysis could be performed. Accordingly, although in Equation 10 above each of the n parameters is multiplied by a corresponding one of the coefficients to form a combined product and then the combined products are added together to provide an estimate of the analyte concentration for a fluid sample for which the analyte concentration is unknown, other methods may be used.

In order to configure a device to determine a concentration of an analyte, the concentrations of non-analyte components may or may not be known. Even if the concentrations of the non-analyte components are not known, there may be a variation in the concentrations across all of the samples and the disclosed methods will account for this variation.

The above embodiments have been described by way of example only, and the described embodiments are to be considered in all respects only as illustrative and not restrictive. It will be appreciated that variations of the described embodiments may be made without the parting from the scope of the claims.

1. A method for configuring a device to determine a concentration of an analyte, the method using a plurality of m fluid samples, each fluid sample of the m fluid samples having a corresponding known analyte concentration, the method comprising:
for each fluid sample of the m fluid samples:
generating an output signal from the fluid sample;
recording values of the output signal over time; and
modelling at least a subset of the recorded values of the output signal using n basis functions to obtain n coefficients, each coefficient being associated with a corresponding basis function, the n basis functions and n coefficients representing the output signal for the subset;
performing a statistical analysis of the m×n coefficients and corresponding known analyte concentrations to determine a set of n parameters from which an analyte concentration can be estimated based on a set of n coefficients obtained for a fluid sample for which the analyte concentration is unknown; and
storing the set of n parameters in a memory of one or more devices.

2. A method according to claim 1, wherein the output signal is a transient current.

3. A method according to claim 1, wherein generating an output signal from the fluid sample comprises applying an input to the fluid sample to generate the output signal, optionally, wherein applying an input to the fluid sample comprises applying a potential difference across the fluid sample.

4. (canceled)

5. A method according to claim 1, wherein the basis functions are orthogonal basis functions, optionally, wherein the basis function functions are orthogonal on the range [0,1], further optionally wherein the basis functions are shifted Legendre polynomials.

6-7. (canceled)

8. A method according to claim 1, wherein n is greater than or equal to 3 and less than or equal to 10 and/or wherein m is greater than or equal to 500 and less than or equal to 1000.

9. A method according to claim 1, wherein the modelling at least a subset of the recorded values of the output signal using n basis functions comprises calculating a least-squares best fit of the recorded values to the n basis functions and/or wherein the performing a statistical analysis of the m×n coefficients and corresponding known analyte concentrations comprises performing a regression analysis of the m×n coefficients and corresponding known analyte concentrations.

10. (canceled)

11. A method according to claim 1, wherein the recording values of the output signal comprises taking time-based measurements of the output signal over time, optionally wherein the recording values of the output signal comprises recording a number of values that is greater than or equal to 100 and is less than or equal to 1000, further optionally, wherein the time-based measurements are recorded at a frequency that is greater than or equal to 10 Hz and less than or equal to 1000 Hz.

12-13. (canceled)

14. A method according to claim 1, wherein modelling at least a subset of the recorded values of the output signal comprises modelling all recorded values or wherein modelling at least a subset of the recorded values of the output signal comprises modelling a portion of the recorded values and optionally wherein modelling at least a subset of the recorded values of the output signal further comprises modelling a second portion of the recorded values.

15-16. (canceled)

17. A method according to claim 1, wherein each fluid sample is a biological fluid sample, optionally wherein the biological fluid sample is a blood sample, an interstitial fluid sample, or a plasma sample.

18. (canceled)

19. A method according to claim 1, wherein each fluid sample of the plurality of m fluid samples comprises a non-analyte component, the presence of which affects the output signal generated for the fluid sample, and wherein there is a variation in the concentration of the non-analyte component across the plurality of m samples.

20. A method according to claim 19, wherein the statistical analysis of the m×n coefficients and corresponding known analyte concentrations corrects for the variation in the concentration of the non-analyte component across the plurality of m samples.

21. A method according to claim 19, wherein, for each fluid sample of the plurality of fluid samples, the concentration of the non-analyte component is known.

22. A method according to claim 19, wherein the non-analyte component comprises red blood cells.

23-24. (canceled)

25. A method according to claim 1, wherein the analyte is one of glucose lactate, glycerol, cholesterol, or a ketone such as β-hydroxybutyrate.

26. An apparatus for configuring a device to determine a concentration of an analyte, the apparatus comprising:
circuitry for generating an output signal from a fluid sample;
a memory storing instructions to perform the method of any preceding claim; and
a processor configured to perform the instructions stored in the memory.

27-31. (canceled)

32. A method of determining a concentration of an analyte in a fluid sample for which the analyte concentration is unknown, the method comprising:
generating an output signal from the fluid sample;
recording values of the output signal over time;
modelling at least a subset of the recorded values of the output signal using n basis functions to obtain n coefficients for the fluid sample, each coefficient being associated with a corresponding basis function, the n basis functions and n coefficients representing the output signal for the subset; and
using a predetermined set of n parameters to estimate the analyte concentration from the n coefficients.

33. A method according to claim 32, wherein the output signal is a transient current.

34. A method according to claim 32, wherein generating an output signal from the fluid sample comprises applying an input to the fluid sample to generate the output signal.

35. A method according to claim 34, wherein applying an input to the fluid sample comprises applying a potential difference across the fluid sample.

36. A method according to claim 32, wherein the using a predetermined set of n parameters to estimate the analyte concentration from the n coefficients comprises:
for each of the n parameters, multiplying the parameter by a corresponding one of the n coefficients to form a combined product; and
adding the combined products to provide an estimate of the analyte concentration.

37-45. (canceled)
